(19) United States
(12) Patent Application Publication
(10) Pub. No.: US 2020/0337568 A1
(43) Pub. Date: Oct. 29, 2020

(54) COMPUTER SYSTEM, DRUG RECOMMENDATION METHOD, AND PROGRAM

(71) Applicant: OPTIM CORPORATION, Saga (JP)

(72) Inventor: Shunji SUGAYA, Tokyo (JP)

(21) Appl. No.: 16/958,884

(22) PCT Filed: Dec. 27, 2017
(86) PCT No.: PCT/JP2017/047010

§ 371 Date: Jun. 29, 2020

(51) Int. Cl. A61B 5/0205; G16H 50/20; G16H 20/10; G16H 40/20; G16H 10/60; G16H 50/70; G16H 70/40; A61B 5/00

(52) U.S. Cl. CPC A61B 5/02055; G16H 50/20; G16H 20/10; G16H 40/20; A61B 5/021; G16H 50/70; G16H 70/40; A61B 5/742; G16H 10/60

(57) ABSTRACT

Provided are a computer system, a drug recommendation method and a program capable of prescribing an appropriate drug for treating an illness. The computer system for recommending a drug corresponding to a diagnosis result of the illness outputs inquiry data for inquiring a user, accepts response data regarding the inquiry data, performs a diagnose on basis of the response data, and learns the diagnosis and a type and dosage of a drug prescribed on basis of the diagnosis in advance and recommends a drug associated with the diagnosis on basis of a result of the learning. In addition to diagnosing, the computer system also learns the type and dosage of the drug on basis of physical condition data, medical history and medication history, etc., of the user in advance and recommends the drug. The computer system notifies a pharmacist capable of prescribing of the recommended drug.

### CROSS-REFERENCE TO RELATED APPLICATIONS

This patent application is a national phase under 35 U.S.C. § 371 of International Patent Application No. PCT/JP2017/047010 filed Dec. 27, 2017, which is hereby incorporated herein by reference in its entirety for all purposes.

### TECHNICAL FIELD

The present disclosure relates to a computer system, a drug recommendation method and a program for recommending a drug corresponding to a diagnosis result of an illness.

### BACKGROUND

In recent years, diagnosing an illness condition of a user has been carried out by an application program installed on terminal apparatuses such as a smart phone, a tablet terminal or the like. As a diagnosis like this, a composition (referring to Patent Literature 1) of diagnosing the illness condition using various information such as vital signs, past medical history, age, etc. of the user is disclosed.

### EXISTING ART DOCUMENT

### Patent Literature

Patent Literature 1: Japanese Laid-open Patent Publication No. 2017-131495

### SUMMARY

### Problems to be Solved

However, in the composition of Patent Literature 1, although the diagnosis of the illness can be performed, it is not easy to determine a drug for treating the illness on basis of a single diagnosis result. The reason is that the drug sometimes does not have sufficient effects due to people, and that it is not easy to prescribe an appropriate drug for treating the illness in the diagnosis of the illness using an existing application program.

An objective of the present disclosure is to provide a computer system, a drug recommendation method and a program capable of prescribing the appropriate drug for treating the illness.

### Solution to the Problem

The present disclosure provides the following solutions.

The present disclosure provides a computer system for recommending a drug corresponding to a diagnosis result of an illness. The computer system includes an output unit, an acceptance unit, a diagnosis unit and a recommendation unit. The output unit is configured to output inquiry data for inquiring a user; the acceptance unit is configured to accept response data regarding to the inquiry data; the diagnosis unit is configured to perform a diagnose on basis of physical condition data included in the response data, wherein the physical condition data includes at least one of a body temperature, an image of affected part, blood pressure, a pulse or a respiration rate of the user; and the recommendation unit is configured to learn a type and dosage of a drug prescribed on basis of the diagnosis and the physical condition data included in the response data in advance, and recommend the drug associated with the diagnosis on basis of a result of the learning.

According to the present disclosure, the computer system for recommending the drug corresponding to the diagnosis result of the illness outputs the inquiry data for inquiring the user, accepts the response data regarding to the inquiry data, performs a diagnose on basis of physical condition data included in the response data, wherein the physical condition data includes at least one of body temperature, an image of affected part, blood pressure, a pulse or respiration rate of the user, and learns a type and dosage of a drug prescribed on basis of the diagnosis and the physical condition data included in the response data in advance and recommends a drug associated with the diagnosis on basis of a result of the learning.

The present disclosure belongs to the category of computer systems, but in other categories such as a method and a program, it still has the same effect and performance as those in this category.

### Effects of the Present Disclosure

The present disclosure can provide a computer system, a drug recommendation method and a program capable of prescribing the appropriate drug for treating the illness.

### BRIEF DESCRIPTION OF THE DRAWINGS

FIG. 1 is a schematic diagram of a drug recommendation system 1.

FIG. 2 is an overall composition diagram of a drug recommendation system 1.

FIG. 3 is a functional block diagram of an information terminal 100.

FIG. 4 is a flowchart illustrating learning processing executed by an information terminal 100.

FIG. 5 is a flowchart illustrating learning diagnosis processing executed by an information terminal 100.

FIG. 6 is a diagram illustrating an example of a state of response data has been accepted.

FIG. 7 is a diagram illustrating an example of a diagnosis result display screen.

FIG. 8 is a diagram illustrating an example of a diagnosis result display screen.

### DETAILED DESCRIPTION

Optimum embodiments for implementing the present disclosure will be described below with reference to the drawings. It is to be noted that the embodiments are merely examples and not intended to limit the scope of the present disclosure.

Summary of Drug Recommendation System 1

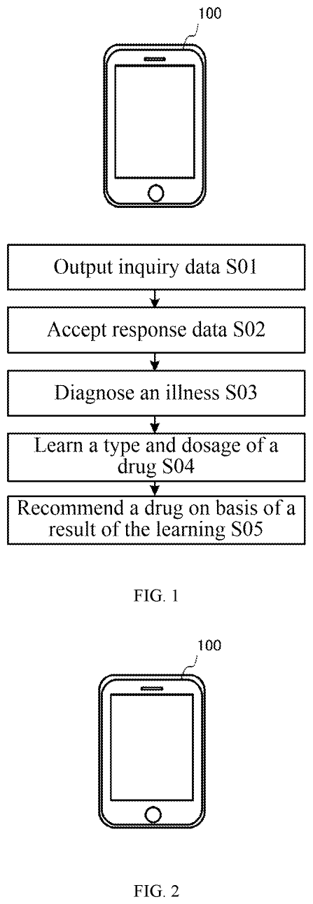

The summary of a preferred embodiment of the present disclosure will be described based on FIG. 1. FIG. 1 is a diagram used for describing the summary of the drug recommendation system 1 as a preferred embodiment of the present disclosure. The drug recommendation system 1 is a computer system which includes an information terminal 100 and is used for recommending a drug corresponding to a diagnosis result of an illness.

It is to be noted that in FIG. 1, the number of information terminals 100 is not limited to one, but may be multiple. In addition, the information terminal 100 is not limited to an actual apparatus, and may be a virtual apparatus. In addition, the drug recommendation system 1 may also have an external apparatus not shown in figure, such as a computer or a terminal apparatus, and may be connected to the information terminal 100 in a manner that data communication can be implemented.

The information terminal 100 is a terminal apparatus capable of diagnosing the illness of a user by using an application program installed in the information terminal 100. In such application, the information terminal 100 acquires various data of the user, such as vital signs, past medical history, medication history, symptoms, and the like, and thereby performing the diagnosis. The information terminal 100 is, for example, a portable phone, a portable information terminal, a tablet terminal or a personal computer, in addition, the information terminal 100 may also be an electrical appliance, such as a netbook terminal, a plate-type terminal, an electronic book terminal, a portable music player or the like; a wearable terminal, such as smart glasses, a head-mounted display or the like; or other devices.

It is to be noted that the diagnosis performed by the above-mentioned application program is not limited to such a composition, and may be modified as appropriate, as long as the corresponding one or more illnesses can be determined on basis of input content accepted from the user as a key point.

The application program installed in the information terminal 100 stores a database related to various kinds of information (illness name, illness condition, symptoms, treatment, and the like) required for diagnosing the illness. The application program diagnoses the illness on basis of the database and the input content accepted from the user, which will be described later. In addition, as will be described later, the application program learns a diagnosis result and the type and dosage of the drug prescribed on basis of the diagnosis in advance, and recommends the drug associated with the diagnosis on basis of the result of the learning. In addition, as will be described later, in addition to learning the diagnosis in advance, the application program further learns the type and dosage of the drug in advance on basis of physical condition data of the user, medical history and medication history of the user included in the response data, and recommends the drug associated with the diagnosis result. In addition, as will be described later, the application program notifies a pharmacist capable of prescribing of the recommended drug.

It is to be noted that the recommended drug by the application program described above is not limited to the composition, and may be modified as appropriate. As a key point, it is only necessary that the diagnosis of the user and the type and dosage of the drug prescribed on basis of the diagnosis can be learned in advance, and the drug associated with the diagnosis can be recommended on basis of the result of the learning.

The information terminal 100 outputs the inquiry data related to the illness to the user (step S01). The information terminal 100 outputs, for example, the inquiry related to the affected part (part or all of the body, such as the head, face, neck, ear, eye, mouth, arm, etc.) as the position where the symptoms occur and the content of the actual symptoms as the inquiry data. At the moment, the information terminal 100 may output the above-mentioned inquiry as a selection input for a plurality of options, or may output a text box for urging the user to input text or sound through a virtual keyboard. The information terminal 100 displays the inquiry data on a display portion of the information terminal 100, thereby outputting the inquiry data.

The information terminal 100 outputs the text box or the selection input for the plurality of options in order to acquire physical condition data of the user matched with the inquiry data.

The information terminal 100 accepts the response data indicating the response to the inquiry data (step S02). The information terminal 100 accepts, for example, the above-mentioned selection input, text input or sound input, and thereby accepts the response data. It is to be noted that the information terminal 100 may also accept an image of the affected part photographed by the user through the photographing apparatus provided in the information terminal 100 as the response data. In this case, the diagnosis described later, the affected part and its symptoms can be diagnosed by image analysis.

When the response data is accepted, the information terminal 100 acquires the physical condition data of the user. The physical condition data refer to, for example, body temperature (body temperature at normal temperature and current body temperature), an image of affected part of the illness (such as allergic illnesses, skin illnesses and infectious illnesses) that are determined to be valid based on the image, the blood pressure, the pulse and the respiration rate. The information terminal 100 may acquire the physical condition data from a device which is communicatively connected to the information terminal 100 and is used for acquiring the physical condition data, or may acquire the physical condition data by accepting the physical condition data matched with the response data for the selection input, text input, or sound input.

The information terminal 100 diagnoses the illness on basis of the accepted response data (step S02). The information terminal 100 references the affected part and the illness corresponding to the symptoms of the affected part in the accepted response data into an illness database having the affected part and the symptoms, thereby determining the illness name of the illness and diagnosing the illness. The illness database records the affected part and the illness name of the illness corresponding to the symptoms of the affected part.

It is to be noted that the information terminal 100 may diagnose the illness in combination with the physical condition data in addition to the response data.

The information terminal 100 determines the type and dosage of the required drug on basis of the diagnosis, and learns the type and dosage of the drug (step S04).

It is to be noted that when the learning is performed, the information terminal 100 may further perform the learning by establishing a correspondence with the physical condition data at the time point at which the diagnosis of the user is performed. In addition, when the learning is performed, the information terminal 100 may further establish a correspondence with the past medical records and medication data of the user to perform the learning. In addition, when the learning is performed, the information terminal 100 may further establish a correspondence with the physical condition data, medical history and the medication data to perform the learning.

In this way, the information terminal 100 learns the type and dosage of drugs appropriate for the user in advance, and uses them in the next diagnosis.

When the user is re-diagnosed, the information terminal 100 outputs the above-mentioned inquiry data, accepts the response data, and diagnoses on basis of the response data, and recommends the drug associated with the diagnosis to the user on basis of the above-mentioned result of the learning (step S05).

It is to be noted that the information terminal 100 may also notify a pharmacist capable of prescribing of the recommended drug. In this case, the information terminal 100 sends data of the drug to an external apparatus held by the corresponding pharmacist, thereby notifying the pharmacist. In addition, the information terminal 100 may perform a video call between the external apparatuses held by the corresponding pharmacist, thereby notifying the pharmacist.

The above is the summary of the drug recommendation system 1.

It is to be noted that the above processing may not necessarily be executed by the information terminal 100 alone. For example, the drug recommendation system 1 may be composed such that the information terminal 100 sends the response data to an external apparatus such as a computer or other terminal apparatus not shown in figure, and the external apparatus performs a diagnosis and outputs a diagnosis result to the information terminal 100. In addition, the drug recommendation system 1 may also be composed such that the information terminal 100 may acquire a result of the learning by performing the above-mentioned learning by the external apparatus. In addition, the drug recommendation system 1 may cause any one or both of the information terminal 100 and the external apparatus to perform any one or more of the above-described processing.

System Composition of a Drug Recommendation System 1

The system composition of the drug recommendation system 1 as a preferred embodiment of the present disclosure will be described based on FIG. 2. FIG. 2 is a diagram of the system composition of the drug recommendation system 1 as a preferred embodiment of the present disclosure. The drug recommendation system 1 is a computer system includes an information terminal 100 and used for recommending a drug corresponding to a diagnosis result of an illness. It is to be noted that the number of information terminals 100 is not limited to one, but may be multiple. In addition, the information terminal 100 is not limited to an actual apparatus, and may be a virtual apparatus. In addition, the drug recommendation system 1 may connected to an external apparatus not shown in figure, such as a computer, a terminal apparatus, or the like through a public network or the like in a manner that data communication can be implemented.

The information terminal 100 is the above terminal apparatus having functions described later.

Description of Functions

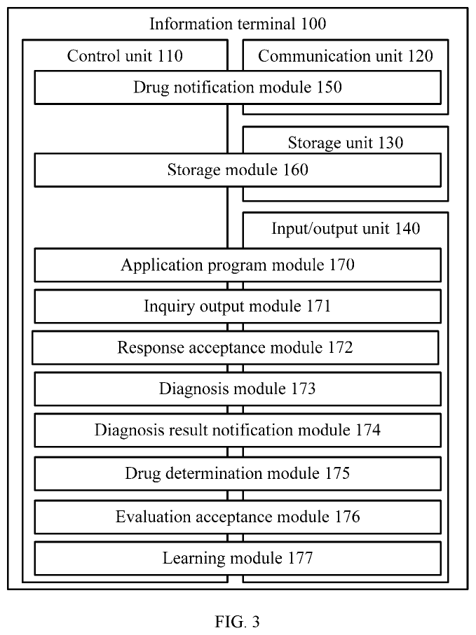

The system functions of the drug recommendation system 1 as a preferred embodiment of the present disclosure will be described based on FIG. 3. FIG. 3 is a functional block diagram of an information terminal 100.

The information terminal 100 is provided with a central processing unit (CPU), a random access memory (RAM), a read only memory (ROM) and the like as a control unit 110, and a device which can communicate with other devices, such as a wireless fidelity (Wi-Fi) component based on IEEE802.11, as a communication unit 120. Furthermore, the information terminal 100 has a storage unit for storing data, such as a hard disk, a semiconductor memory, a recording medium, a memory card and the like, as a storage unit 130. The information terminal 100 stores an illness database described later in the storage unit 130. Furthermore, the information terminal 100 has a display unit, for outputting and displaying data or images, controlled by the control unit 110, an input unit such as a touch panel, a keyboard or a mouse for receiving input of a user and other various components as an input/output unit 140.

In the information terminal 100, the control unit 110 reads specific programs and cooperates with the communication unit 120 to implement a drug notification module 150. Furthermore, in the information terminal 100, the control unit 110 reads the specific programs and cooperates with the storage unit 130 to implement a storage module 160. Furthermore, in the information terminal 100, the control unit 110 reads the specific programs and cooperates with the input/output unit 140 to implement an application program module 170, an inquiry output module 171, a response acceptance module 172, a diagnosis module 173, a diagnosis result notification module 174, a drug determination module 175, an evaluation acceptance module 176, and a learning module 177.

Learning Processing

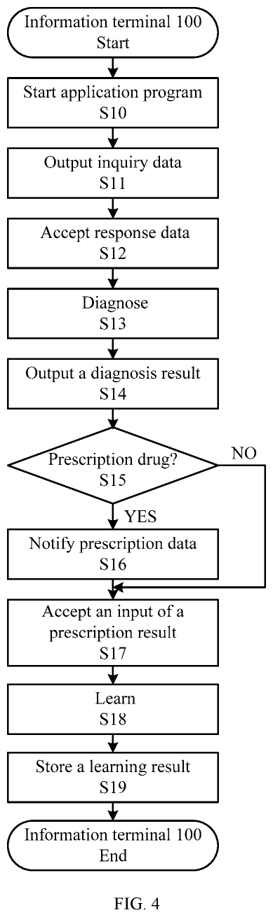

The learning processing executed by the drug recommendation system 1 will be described based on FIG. 4. FIG. 4 is a flowchart illustrating learning processing executed by an information terminal 100. Processing performed by the above modules will be described in conjunction with the processing.

First, the application program module 170 starts a diagnostic application program (step S10). In step S10, the application program module 170 accepts a start-up input from the user implemented by a tap input, a sound input, or the like, and starts a corresponding diagnostic application program. In the following processing, a state of the actual processing executed by the application will be described.

The inquiry output module 171 outputs a plurality of options, inquiries, text boxes, and the like related to the affected part and the symptoms corresponding to the affected part as the inquiry data (step S11), where the text boxes accept the affected part and the symptoms corresponding to the affected part directly input by the user. In step S11, the inquiry output module 171 displays the inquiry data on a display unit. The inquiry data includes an option or a text box for acquiring the physical condition data of the user. The physical condition data refers to, for example, body temperature (body temperature at normal temperature and current body temperature), an image of affected part of the illness (such as allergic illnesses, skin illnesses or infectious illnesses) for which the determination based on the image is valid, the blood pressure, the pulse and the respiration rate.

It is to be noted that the inquiry output module 171 may also output the inquiry data by sound output or the like.

The response acceptance module 172 accepts the response to the inquiry data as the response data (step S12). In step S12, the response acceptance module 172 accepts the selection input of the above options, the text input achieved by the virtual keyboard, the sound input achieved by the sound from the user, or the like, and thereby accepts the response data. The response acceptance module 172 accepts the above-mentioned physical condition data to acquire the physical condition data as the response data. The response acceptance module 172 may accept the physical condition data through the selection input, text input, or sound input from the user, or may also accept various data measured by the external apparatus communicatively connected to acquire the physical condition data, such as a thermometer, a photographing apparatus, a sphygmomanometer, and a respirometer.

It is to be noted that the response acceptance module 172 may also accept the image of the affected part photographed by the photographing apparatus or the like as the response data. In this case, during a processing of the diagnosis described later, the information terminal 100 performs the image analysis, determines the affected part and the symptoms of the affected part, and performs the diagnosis based on the determined result.

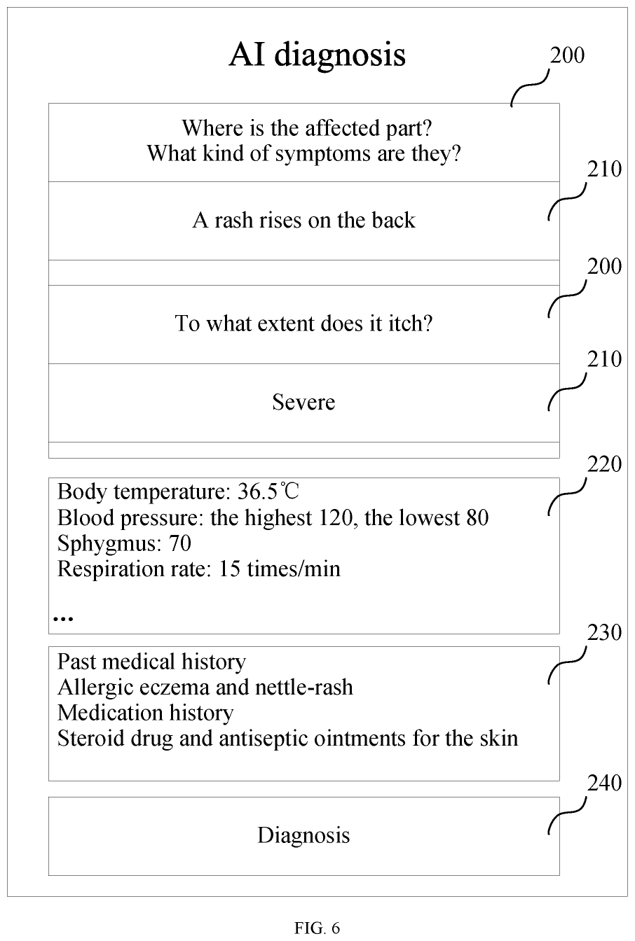

The response data accepted by the response acceptance module 172 will be described based on FIG. 6. FIG. 6 is diagram illustrating an example of a state of response data has been accepted. As shown in FIG. 6, the inquiry output module 171 displays an inquiry display area 200, and the response acceptance module 172 displays a response acceptance area 210, a physical condition acceptance area 220, an acceptance area 230 of the medical history and medication history, and a diagnosis icon 240. The inquiry display area 200 is an area displayed the above-mentioned inquiries. As shown in FIG. 6, the inquiry output module 171 displays in the inquiry display area 200: “where is the affected part?” “what kind of symptom is it?” and “to what extent does it itch?”. In the response acceptance area 210, the “a rash rises on the back” and “severe” input by the user are displayed. The inquiry output module 171 additionally displays new inquiry content in the inquiry display area 200 based on the response accepted from the user. Specifically, first, the inquiry output module 171 displays an inquiry of the affected part and the symptoms of the affected part in the inquiry display area 200. In a case where the inquiry acceptance module 172 accepts the “rash on the back” input by the user for the inquiry, the inquiry acceptance module 172 performs the text analysis, thereby confirming the input content and determining the affected part and the symptoms. In a case where the inquiry for determining the actual illness is required based on the determined result, the inquiry output module 171 displays a further inquiry in the inquiry display area 200. In the embodiment, “to what extent is it itchy?” is equivalent to additionally displaying the new inquiry content. The response acceptance module 172 displays the “serious” input accepted as the response of the inquiry in the response acceptance area 210. The response acceptance module 172 accepts the above-mentioned physical condition data, and the above-mentioned physical condition data is displayed in the response acceptance area 210. The response acceptance module 172 displays respective accepted values of the “body temperature, blood pressure, pulse, respiration rate, etc.”. The response acceptance module 172 accepts the past medical history and medication history of the user, and the past medical history and medication history of the user are displayed in the acceptance area 230 of the medical history and medication history. The acceptance area 230 of the medical history and medication history is not limited to input from the user, but may also display a name of the illness and a name and dosage of the drug prescribed for the illness as a result of the past diagnosis by the diagnostic application program. The response acceptance module 172 accepts an input operation to the diagnostic icon 240, thereby detecting the completion of the input, and the diagnosis module 173 executes the diagnosis described later.

The diagnosis module 173 performs a diagnosis based on the accepted response data (step S13).

In step S13, the diagnosis module 173 diagnoses the illness corresponding to the affected part and the symptoms of the affected part in the accepted response data, and the type and dosage of the drug for the illness. At the moment, in the case where the learning result of the same symptoms or similar symptoms has been diagnosed exists so far, the learning diagnosis processing described later is executed. On the other hand, in a case where no learning result exists, the diagnosis module 173 diagnoses the illness based on an illness database in which the affected part and the symptoms, the illness name and a processing method (the type and dosage of the drug) of the corresponding illness, and the risk degree of the illness, with an established correspondence among them, are entered. The illness database is pre-stored in the storage module 160.

Illness Database

The illness database pre-stored in the storage module 160 will be described. The storage module 160 pre-stores the illness database acquired in advance from an external database, the external apparatus, or the like. The illness database may also be an illness database stored in the diagnostic application program. As mentioned above, the illness database establishes a correspondence among the affected part and the symptoms of the affected part, the illness name of the actual illness, the processing method (e.g., therapeutic drugs, therapies) and the risk degree (e.g., a high value for illnesses requiring early treatment, a moderate value for illnesses at risk in the case of chronic treatment, and a low value for illnesses that are naturally cured).

In the above-mentioned example, the diagnosis module 173 determines, with reference to the illness database, the illness corresponding to the affected part and the symptoms, based on the response data that the affected part is “the back”, the symptom is “rash”, and the itching degree is “severe”. The diagnosis module 173 determines the corresponding illness as “allergic eczema” at this time. At the moment, in a case where a plurality of illnesses are determined, the illness with the highest possibility is judged as the diagnosis result. It is to be noted that in a case where a plurality of illnesses are determined, the diagnosis module 173 may not judge one illness as the diagnosis result, but may judge the plurality of illnesses as the diagnosis result. In this case, the possibilities of the plurality of illnesses is judged.

The diagnosis result notification module 174 outputs the diagnosis result (step S14). In step S14, the diagnosis result notification module 174 displays the diagnosis result on the display unit, thereby outputting the diagnosis result to notify the user.

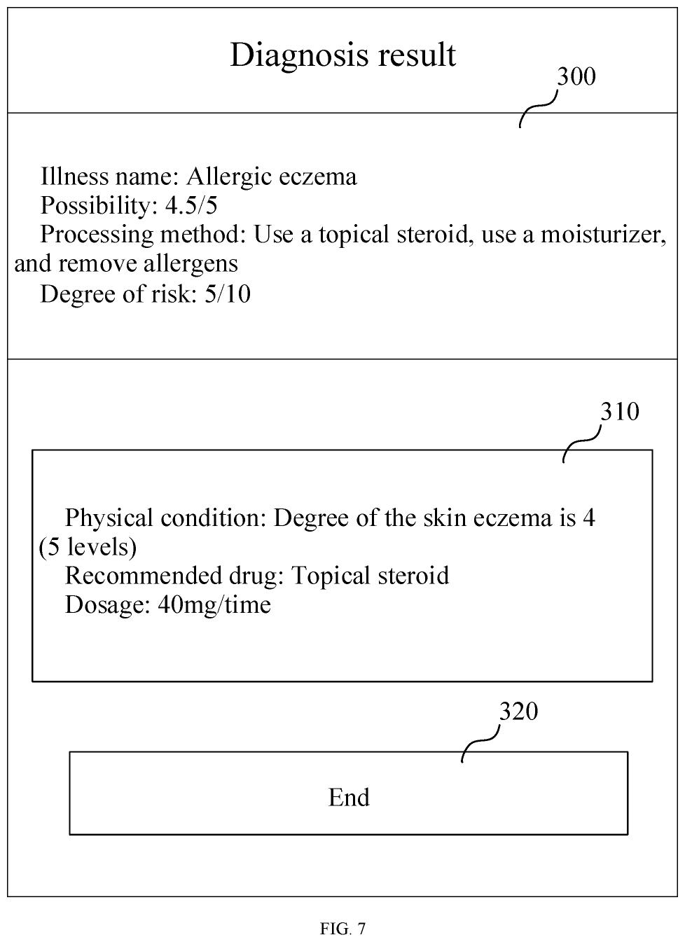

The diagnosis result notification module 174 displays the diagnosis result based on FIG. 7. An example of a diagnosis result display screen will be described. FIG. 7 is diagram illustrating an example of a diagnosis result display screen displayed by the diagnosis result notification module 174. As shown in FIG. 7, the diagnosis result notification module 174 displays a diagnosis result display area 300, a display area 310 of the physical condition and a drug, and an ending icon 320, as a diagnosis result display screen. The diagnosis result display area 300 is an area for displaying the diagnosis result. The display area 310 of the physical condition and the drug is an area for displaying a degree of the illness condition based on the physical condition data, and the name and dosage of the drug to be prescribed. The diagnosis result notification module 174 displays the result of this diagnosis in the diagnosis result display area 300. As shown in FIG. 7, the illness name of the illness is displayed, and the possibility of the illness is displayed by an evaluation with 5 levels; and the processing method of the illness is displayed, and the risk degree of the illness is displayed by an evaluation with 10 levels. The diagnosis result notification module 174 displays the name and dosage of the drug to be prescribed based on the diagnostic illness in the display area 310 of the physical condition and the drug. In FIG. 7, the degree of the skin eczema is displayed by an evaluation with 5 levels, and the recommended drug and the dosage of the recommended drug for the illness are displayed. The diagnosis result notification module 174 accepts an input operation to the ending icon 320, thereby detecting the completion of the display, and ending the display of the determined diagnosis result.

The diagnosis result notification module 174 recommends the drug associated with the diagnosis to the user.

The drug determination module 175 determines whether the drug outputted for this time is a prescription drug (step S15). In step S15, the drug determination module 175 determines whether the drug outputted for this time is the prescription drug based on the name of the drug. In a case where the drug determination module 175 determines that the drug outputted for this time is not the prescription drug (no in step S15), the processing of step S17 described later is executed.

On the other hand, in step S15, in a case where the drug determination module 175 determines that the drug outputted for this time is the prescription drug (yes in step S15), the drug notification module 150 notifies the pharmacist who can prescribe the drug of prescription data indicating the name and dosage of the drug (step S16). In step 16, the drug notification module 150 outputs the prescription data to a terminal apparatus not shown in figure held by the pharmacist to be targeted and displays the prescription data. The pharmacist prepares the required drug and dosage based on the prescription data.

It is to be noted that in the case where the drug is a special drug, such as a case where an interview with the pharmacist is required, the drug notification module 150 may, when outputting the prescription data, call the terminal apparatus through its own telephone function to perform a normal call, a video call, or the like. Furthermore, even in a case where the drug is not the prescription drug, the drug notification module 150 may also notify the prescription data to the pharmacist to be targeted who processes the drug.

The evaluation acceptance module 176 accepts an input of a prescription result of how the symptoms are changed by the drug based on this diagnosis result (step S17). In step S17, the evaluation acceptance module 176 accepts a positive evaluation such as the cure of the symptoms, a negative evaluation such as the absence of change or deterioration of the symptoms, and a neutral evaluation such as not knowing whether the symptoms have improved as a result of using the notified drug. At the moment, similarly to the above-mentioned response data, the evaluation acceptance module 176 accepts the selection input for the options, text input, sound input, etc.

It is to be noted that similarly to the response data, the evaluation acceptance module 176 also accepts the image of the affected part as a prescription result. In this case, the evaluation acceptance module 176 may perform the image analysis for the image of the affected part, compare the image of the affected part before the drug is used with the image of the affected part after the drug is used, and thereby determine the above-mentioned evaluation of the symptoms, and thereby accept the evaluation.

The learning module 177 learns the diagnosis result, the type and dosage of the prescribed drug, and the physical condition data of the user, the past medical history and medication history of the user, and the evaluation of the prescription result included in the response data (step S18). In step S18, the learning module 177 learns the type and dosage of the drug, the physical condition data, the past medical history and the medication history which have an established correspondence with positive evaluation of the prescription result as correct response data. Furthermore, the learning module 177 learns the data having an established correspondence with the neutral or negative evaluation as incorrect response data.

It is to be noted that the learning module 177 may perform learning based on any one or more of combinations of the above data. For example, the learning module 177 may learn by establishing a correspondence between the diagnosis result and the type and dosage of the prescribed drug, may also learn by establishing a correspondence among the diagnosis result, the type and dosage of the prescribed drug, and the physical condition data, or may also learn by establishing a correspondence among the diagnosis result, the type and dosage of the prescribed drug, and the medical history and medication history of the user, or may also learn by other combination.

The storage module 160 stores result of the learning (step S19). In step S19, the storage module 160 stores correct response data and incorrect response data as the results of the learning, respectively.

The above is the learning processing.

Learning Diagnosis Processing

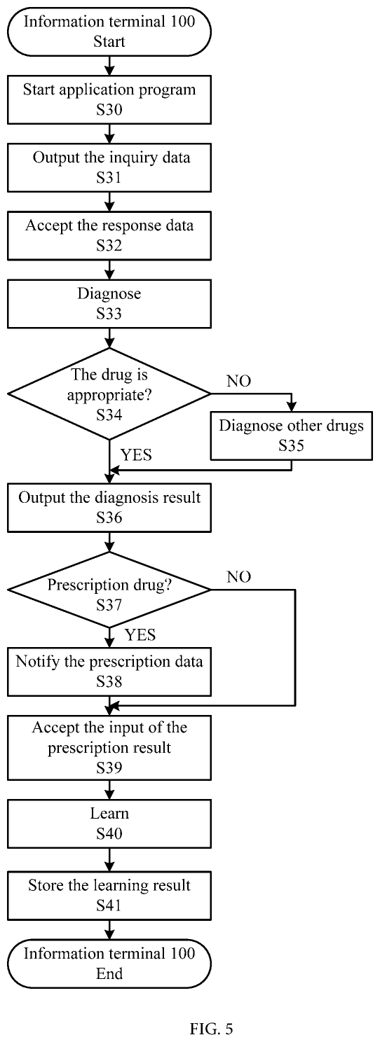

The learning diagnosis processing executed by the drug recommendation system 1 will be described based on FIG. 5. FIG. 5 is a flowchart illustrating learning diagnosis processing executed by an information terminal 100. Processing performed by the above modules will be described in conjunction with the processing. It is to be noted that the detail in the processing similar to the above-mentioned learning processing will be omitted.

Similar to the above-mentioned diagnostic processing, the information terminal 100 executes the processing of starting the diagnostic application program, outputting the inquiry data, and accepting the response data (steps S30 to S32).

The diagnosis module 173 performs the diagnosis based on the accepted response data (step S33). In step S33, the diagnosis module 173 diagnoses the illness corresponding to the affected part and the symptoms of the affected part in the accepted response data, and the type and dosage of the drug for the illness. At the moment, the diagnosis module 173 uses the learning data stored in the storage module 160 when diagnosing the type and dosage of the drug.

The drug determination module 175 determines whether the type and dosage of the drug determined by the current diagnosis result are appropriate based on the type and dosage of the drug determined by the current diagnosis result and the type and dosage of the drug in the learning data (step S34). In step S34, the drug determination module 175 determines whether the type and dosage of the drug determined by the current diagnosis result consistent with or approximate to the correct response data, and thereby determines whether the drug is appropriate. Specifically, being consistent with or approximate to the correct response data refers to: the types and dosage of drug are the same; the types of drugs are the same, but the dosage of the drugs is different; or the types of drugs are different, but the drugs are a generic drug and other drug that can expect substantially the same effect.

In step S34, in a case where the drug determination module 175 determines that the drug is inappropriate (no in step S34), the drug determination module 175 determines that the drug determined by the current diagnosis result is inappropriate to the user, and the diagnosis module 173 diagnoses the other drug (step S35). In step S35, the diagnosis module 173 consults the illness database and the like described above, thereby diagnosing another drug having a similar effect to the drug determined by the diagnosis result.

The diagnosis result notification module 174 outputs the diagnosed illness and the type and dosage of the re-diagnosed drug as the diagnosis result (step S36).

On the other hand, in step S34, in a case where the drug determination module 175 determines that it is appropriate (yes in step S34), the diagnosis result notification module 174 outputs the diagnosed illness and the type and dosage of the drug as the diagnosis result (step S36).

In step S36, the diagnosis result notification module 174 displays the diagnosis result on the display unit, and outputs the diagnosis result to notify the user.

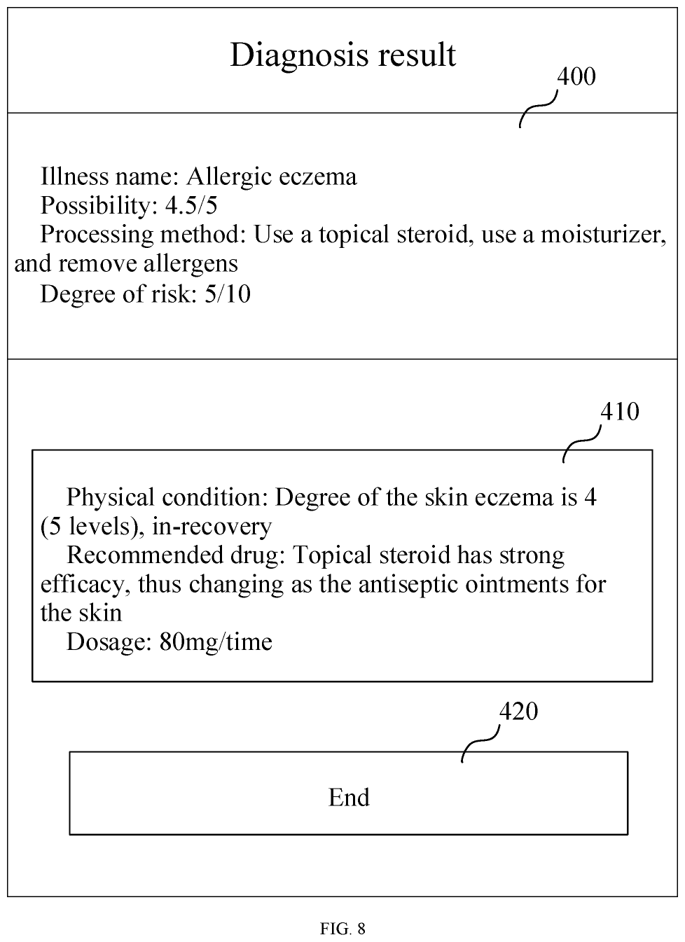

The diagnosis result notification module 174 displays the diagnosis result based on FIG. 8. An example of a diagnosis result display screen will be described. FIG. 8 is diagram illustrating an example of a diagnosis result display screen displayed by the diagnosis result notification module 174. As shown in FIG. 8, the diagnosis result notification module 174 displays a diagnosis result display area 400, a display area 410 of the physical condition and a drug, and an ending icon 420, as a diagnosis result display screen. The diagnosis result display area 400 is an area for displaying the diagnosis result. The display area 410 of the physical condition and the drug is an area for displaying a degree of the illness condition based on the physical condition data and the name and dosage of the drug to be prescribed. The diagnosis result notification module 174 displays the result of this diagnosis in the diagnosis result display area 400. As shown in FIG. 8, the illness name of the illness is displayed, the possibility of the illness is displayed by an evaluation with 5 levels, and the processing method of the illness is displayed, and the risk degree of the illness is displayed by an evaluation with 10 levels. The diagnosis result notification module 174 displays the name and dosage of the drug to be prescribed based on the diagnostic illness in the display area 410 of the physical condition and the drug. In FIG. 8, the degree of the skin eczema is displayed by an evaluation with 5 levels, and the recommended drug and the dosage of the recommended drug for the illness are displayed. For the recommended drug and the dosage of the recommended drug, the content is determined by the learning result based on the learning data and the physical condition data of the user. In addition, for the recommended drug and the dosage of the recommended drug, the content is determined by the learning result based on the learning data and the past medical history and medication data of the user. The diagnosis result notification module 174 accepts an input operation to the ending icon 420, thereby detecting the completion of the display, and ending the display of the determining diagnosis result.

In this way, the diagnosis result notification module 174 recommends the drug associated with the diagnosis according to the learning results of the drug corresponding to any one of the physical condition and the past medical history and medication data of the diagnosed user, or corresponding to both of the physical condition and the past medical history and medication data of the diagnosed user.

For the information terminal 100, the subsequent processing is the same as the processing after step S15 of the above-mentioned learning processing, and therefore a simple description will be performed.

The drug determination module 175 determines whether the drug outputted for this time is the prescription drug (step S37). The processing of step S37 is the same as the processing of step S15 described above. In a case where the drug determination module 175 determines that the drug outputted for this time is not the prescription drug (no in step S37), the processing of step S39 described later is executed.

On the other hand, in step S37, in a case where the drug determination module 175 determines that the drug outputted for this time is the prescription drug (yes in step S37), the drug notification module 150 notifies the pharmacist who can prescribe the drug of prescription data indicating the name and dosage of the drug (step S38). The processing of step S38 is the same as the processing of step S16 described above.

The evaluation acceptance module 176 accepts an input of a prescription result of how the symptoms are changed by the drug based on the diagnosis result (step S39). The processing of step S39 is the same as the processing of step S17 described above.

The learning module 177 learns the diagnosis result, the type and dosage of the prescribed drug, and the physical condition data of the user, the past medical history and medication history of the user, and the evaluation of the prescription result included in the response data (step S40). The processing of step S40 is the same as the processing of step S18 described above.

The storage module 160 stores result of the learning (step S41). The processing of step S41 is the same as the processing of step S19 described above.

The above is the learning diagnosis processing.

It is to be noted that the above processing may not necessarily be executed by the information terminal 100 alone. For example, the drug recommendation system 1 may also be composed such that the information terminal 100 sends the response data to an external apparatus such as a computer or other terminal apparatus not shown in figure, and the external apparatus performs a diagnosis and outputs a diagnosis result to the information terminal 100. In addition, the drug recommendation system 1 may also cause any one or both of the information terminal 100 and the external apparatus to perform any one or more pieces of the above-described processing.

The above units and functions are implemented by reading and executing specified programs by a computer (including a CPU, an information processing apparatus and various terminals). The programs, for example, are provided by a solution provided by a computer via a network (i.e., software as a service (SaaS)). Furthermore, the programs are provided a solution recorded in a computer-readable recording medium such as a floppy disk, a compact disk (CD) (such as a compact disc read-only memory (CD-ROM)), and a digital versatile disc (DVD) (such as a DVD-ROM and a DVD random access memory (DVD-RAM)). In this case, the computer reads the program from the storage medium and sends the program to an internal storage device or an external storage device so that the program is stored; then the computer executes the program. Furthermore, the programs may also be recorded in advance on a storage apparatus (recording medium) such as a magnetic disk, an optical disk or a magneto-optical disk, and provided from the storage apparatus for the computer via a communication line.

The embodiments of the present disclosure have been described above, but the present disclosure is not limited to the above embodiments. In addition, the effects described in the embodiments of the present disclosure are merely illustrative of the most appropriate effects produced by the present disclosure, and the effects of the present disclosure are not limited to the effects described in the embodiments of the present disclosure.

### REFERENCE LIST

1 drug recommendation system
100 information terminal.

1-6. (canceled)

7. A computer system, for recommending a drug corresponding to a diagnosis result of an illness, comprising:
an output unit, configured to output inquiry data for inquiring a user;
an acceptance unit, configured to accept response data regarding to the inquiry data;
a diagnosis unit, configured to perform a diagnose on basis of physical condition data included in the response data, wherein the physical condition data includes at least one of a body temperature, an image of affected part, blood pressure, a pulse or a respiration rate of the user; and
a recommendation unit, configured to learn the diagnosis and a type and dosage of a drug prescribed on basis of the diagnosis and the physical condition data included in the response data in advance, and recommend the drug associated with the diagnosis on basis of a result of the learning.

8. The computer system of claim 7, wherein
in addition to learning the diagnosis in advance, the recommendation unit is further configured to learn the type and dosage of the drug in advance on basis of medical history and medication history of the user, and recommend the drug associated with the diagnosis.

9. The computer system of claim 7, further comprising:
a notification unit, configured to notify a pharmacist capable of prescribing of the drug recommended by the recommendation unit.

10. A drug recommendation method, performed by a computer system for recommending a drug corresponding to a diagnosis result of an illness, comprising:
outputting inquiry data for inquiring a user;
accepting response data regarding to the inquiry data;
performing a diagnose on basis of physical condition data included in the response data, wherein the physical condition data includes at least one of body temperature, an image of affected part, blood pressure, a pulse or respiration rate of the user; and
learning the diagnosis and a type and dosage of a drug prescribed on basis of the diagnosis and the physical condition data included in the response data in advance, and recommending the drug associated with the diagnosis on basis of a result of the learning.

11. A non-transitory computer readable program, used for causing a computer system for recommending a drug corresponding to a diagnosis result of an illness to perform the following steps:
outputting inquiry data for inquiring a user;
accepting response data regarding to the inquiry data;
performing a diagnose on basis of physical condition data included in the response data, wherein the physical condition data includes at least one of body temperature, an image of affected part, blood pressure, a pulse or respiration rate of the user; and
learning the diagnosis and a type and dosage of a drug prescribed on basis of the diagnosis and the physical condition data included in the response data in advance, and recommending the drug associated with the diagnosis on basis of a result of the learning.
